(19) United States
(12) Patent Application Publication
(10) Pub. No.: US 2021/0244836 A1
(43) Pub. Date: Aug. 12, 2021

(54) UV STERILIZER

(71) Applicant: POINT ENGINEERING CO., LTD., Asan (KR)

(72) Inventors: Bum Mo AHN, Suwon (KR); Young Woon JEON, Ansan (KR); Min Woo CHOI, Pyeongtaek (KR)

(21) Appl. No.: 17/167,938

(22) Filed: Feb. 4, 2021

(30) Foreign Application Priority Data
Feb. 11, 2020 (KR) 10-2020-0016371

(51) Int. Cl. A61L 2/10

(52) U.S. Cl. CPC A61L 2/10; A61L 2202/11

(57) ABSTRACT

Proposed is a UV sterilizer. In a UV sterilizer according to an embodiment of the present disclosure, a light source module that emits ultraviolet (UV) light is provided between an upper body and a lower body where a fluid flows, thereby sterilizing a fluid by irradiating the flowing fluid with UV light.

### CROSS REFERENCE TO RELATED APPLICATION

The present application claims priority to Korean Patent Application No. 10-2020-0016371, filed Feb. 11, 2020, the entire contents of which is incorporated herein for all purposes by this reference.

### BACKGROUND OF THE INVENTION

### Field of the Invention

The present disclosure relates generally to a UV sterilizer and, more particularly, to a UV sterilizer for sterilizing a fluid by having a light source module emitting ultraviolet (UV) light in a flow path along which the fluid flows.

### Description of the Related Art

Sterilization refers to the complete elimination of all living organisms, including viruses, bacteria, fungi, microorganisms, mites, and the like. Common methods of sterilization include a physical method including a heating method for sterilizing an object using heat or steam, and a chemical method for sterilizing an object using a sterilizing agent or a sterilizing gas.

In recent years, as the awareness of bacteria and the interest in health have increased among the general public, there has been a growing interest in devices or methods that can facilitate sterilization.

The physical method, which is generally used to eliminate viruses, bacteria, microorganisms, or mites, includes a method in which microorganisms or the like are eliminated by applying heat or steam having a high temperature to an object to be sterilized. However, this method is problematic in that time and fuel to secure a high temperature are required and thus it takes a long time to sterilize.

The chemical method using a sterilizing agent or a sterilizing gas is problematic in that the sterilizing agent is generally made of a chemical substance and thus a user may be exposed to toxic substances in the process of using the sterilizing agent or the sterilizing gas.

In an effort to solve the above problems, there has been developed a sterilization method using ultraviolet radiation which is a physical sterilization method.

As a patent that describes sterilization of a fluid using such ultraviolet irradiation, Korean Patent Application Publication No. 10-2017-0028772 (hereinafter referred to as ‘Patent Document 1’) is known.

In Patent Document 1, an ultraviolet light-emitting diode (UV LED) that irradiates a pipe with ultraviolet (UV) light is installed on the outside of the pipe to sterilize a fluid flowing inside the pipe by irradiation by UV light. In this case, the pipe is made of a material that allows easy transmission of UV light.

However, Patent Document 1 is problematic in that since the irradiation by UV light is performed outside the pipe, a sterilization effect by the UV LED may be slightly deteriorated even if UV light is easily transmitted through the pipe.

The foregoing is intended merely to aid in the understanding of the background of the present disclosure, and is not intended to mean that the present disclosure falls within the purview of the related art that is already known to those skilled in the art.

### DOCUMENTS OF RELATED ART

(Patent document 1) Korean Patent Application Publication No. 10-2017-0028772

### SUMMARY OF THE INVENTION

Accordingly, the present disclosure has been made keeping in mind the above problems occurring in the related art, and an objective of the present disclosure is to provide a UV sterilizer capable of sterilizing a fluid by irradiating the fluid flowing in a flow path with ultraviolet (UV) light by having a light source module in the flow path.

In order to achieve the above objective, according to one aspect of the present disclosure, there is provided a UV sterilizer, including: an upper body having an upper flow path; a lower body having a lower flow path; and a light source module provided between the upper body and the lower body, wherein the light source module may include: a UV light source; and a support supporting the UV light source, and having a through flow path allowing communication between the upper flow path and the lower flow path.

Furthermore, the UV sterilizer may further include: a fluid guide provided on at least one of upper and lower portions of the light source module, wherein the fluid guide may include: a side wall; and an inner flange with an opening extending inwardly from the sidewall.

Furthermore, the UV sterilizer may further include: a flow resistor provided in the upper flow path, wherein the flow resistor may include: a base plate; and an extension flow path extending upwardly from the base plate, and having a plurality of holes formed in a side surface thereof.

Furthermore, an upper fastening portion fastened to an upper portion of the light source module may be provided at a lower portion of the upper body, and a lower fastening portion fastened to a lower portion of the light source module may be provided at an upper portion of the lower body.

Furthermore, the through flow path may include a plurality of through flow paths, and the through flow paths may be formed in arc shapes spaced apart from each other.

Furthermore, the light source module may include a plurality light source modules, and a fluid guide may be provided between the light source modules.

The UV sterilizer according to the present disclosure as described above has the following effects.

By providing the light source module having the UV light source in a flow path along which the fluid flows, it is possible to sterilize the flowing fluid by irradiation by UV light.

In addition, by providing the plurality of light source modules in a flow path, it is possible to sterilize the fluid flowing along the flow path a plurality of times through the plurality of light source modules.

In addition, by installing the fluid guide and the flow resistor to guide the fluid to an upper portion of the light source module, it is possible to increase sterilization efficiency by UV light.

In addition, by providing the fluid guide at each of upper and lower portions of the light source module, the fluid is guided to a central portion of a lower surface of the light source module so that the fluid flows from the central portion of the lower surface of the light source module to peripheral portions thereof, thereby making it possible to effectively cool the light source module.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a view illustrating a UV sterilizer according to a first embodiment of the present disclosure;

FIG. 2 is a plan view of a UV light source;

FIG. 3 is a sectional view of the UV light source.

FIGS. 4A and 4B are views illustrating a UV sterilizer according to a second embodiment of the present disclosure;

FIG. 5 is a view illustrating a UV sterilizer according to a third embodiment of the present disclosure; and

FIG. 6 is a view illustrating a state in which a plurality of light source modules is provided in the UV sterilizer according to the third embodiment of the present disclosure.

### DETAILED DESCRIPTION OF THE INVENTION

Contents of the description below merely exemplify the principle of the present disclosure. Therefore, those of ordinary skill in the art may implement the theory of the present disclosure and invent various apparatuses which are included within the concept and the scope of the present disclosure even though it is not clearly explained or illustrated in the description. Furthermore, in principle, all the conditional terms and embodiments listed in this description are clearly intended for the purpose of understanding the concept of the present disclosure, and one should understand that this present disclosure is not limited to such specifically listed exemplary embodiments and conditions.

The above described objectives, features, and advantages will be more apparent through the following detailed description related to the accompanying drawings, and thus those of ordinary skill in the art may easily implement the technical spirit of the present disclosure.

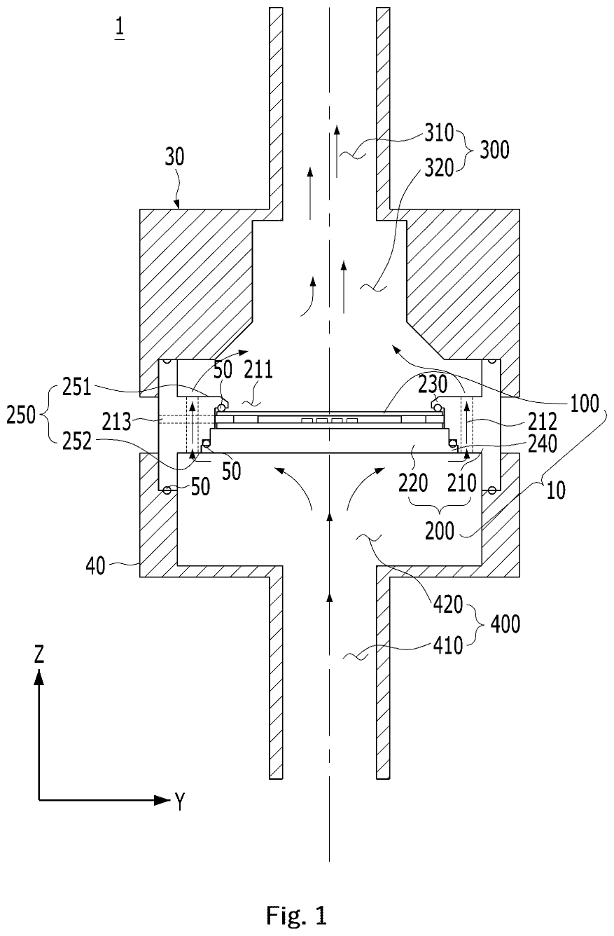

Hereinbelow, a UV sterilizer according to a first embodiment of the present disclosure will be described with reference to FIGS. 1 to 3.

Referring to FIGS. 1 to 3, the UV sterilizer 1 may include an upper body 30 provided with an upper flow path 300, a lower body 40 provided with a lower flow path 400, and a light source module 10 provided between the upper body 30 and the lower body 40, and the light source module 10 may include a UV light source 100 and a support 200 supporting the UV light source 100 and provided with a through flow path 212 allowing communication between the upper flow path 300 and the lower flow path 400.

The upper body 30, the lower body 40, and the light source module 10 may be formed in a polygonal shape such as a circle or a square, and in the following description, the upper body 30, the lower body 40, and the light source module 10 will be illustrated and described as having a circular shape as an example.

As illustrated in FIG. 1, the upper body 30 may be provided on the light source module 10, and the upper flow path 300 may be provided in the upper body 30. The upper flow path 300 may be composed of a first upper flow path 310 and a second upper flow path 320.

The first upper flow path 310 may have a first side open, and a second side in communication with the second upper flow path 320. The first upper flow path 310 may have an outer diameter and an inner diameter smaller than those of the second upper flow path 320.

The second upper flow path 320 may have a first side in communication with the first upper flow path 310, and a second side open. The second upper flow path 320 may have an outer diameter and an inner diameter larger than those of the first upper flow path 310.

The second upper flow path 320 may have an inner shape formed so that the inner diameter of the second upper flow path 320 gradually decreases from bottom to top thereof. The width of the inner diameter of the second upper flow path 300 may be smallest at the top and the largest at the bottom. By the shape of the second upper flow path 300, when a fluid flows into the upper body 30 through the light source module 10, the flow of the fluid flowing in may be guided to an upper portion of the light source module 10, thereby improving a UV exposure time for the flowing fluid.

The lower body 40 may be provided under the light source module 10, and the lower flow path 400 may be provided in the lower body 40. The lower flow path 400 may be composed of a first lower flow path 410 and a second lower flow path 420.

The first lower flow path 410 may have a first side in communication with the second lower flow path 420, and a second side open. The first lower flow path 410 may have an outer diameter and an inner diameter smaller than those of the second lower flow path 420.

The second lower flow path 420 may have a first side open, and a second side in communication with the first lower flow path 410. The second lower flow path 420 may have an outer diameter and an inner diameter larger than those of the first lower flow path 410.

The light source module 10 may be provided between the upper body 30 and the lower body 40. The light source module 10 may include the UV light source 100, and the support 200 supporting the UV light source 100 and provided with the through flow path 212 allowing communication between the upper flow path 300 and the lower flow path 400.

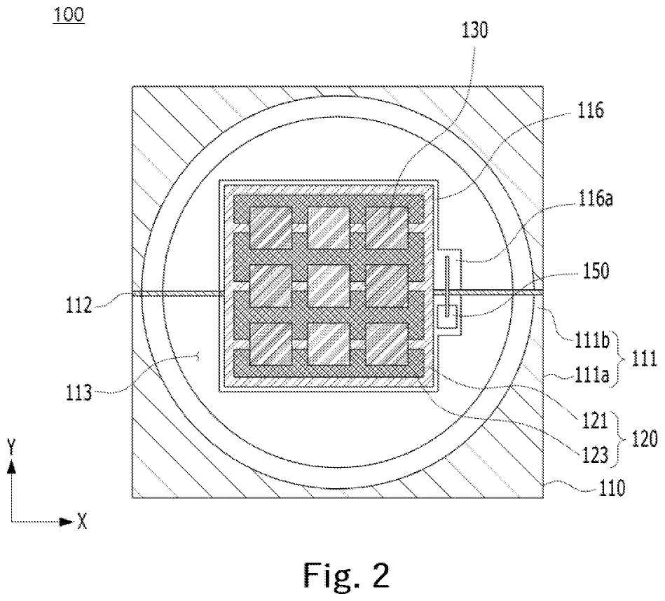

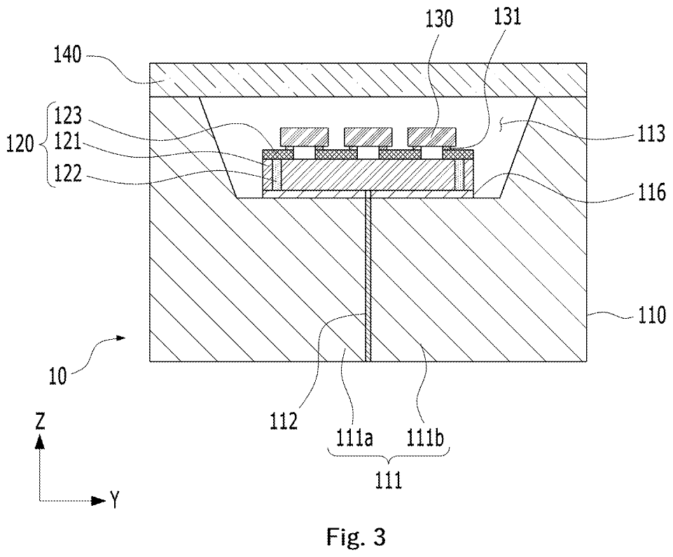

As illustrated in FIGS. 2 and 3, the UV light source 100 may include a main substrate 110, a sub-substrate 120 provided at the main substrate 110, an optical element 130 mounted on the sub-substrate 120, and a light transmission member 140 provided above the main substrate 110, the sub-substrate 120, and the optical element 130.

The main substrate 110 includes a plurality of metal bodies 111 and a vertical insulation part 112 provided between the metal bodies 111. The metal bodies 111 may be made of a metal plate having excellent electrical conductivity and thermal conductivity. For example, the metal bodies 111 may be made of any one selected from aluminum, an aluminum alloy, copper, a copper alloy, iron, an iron alloy, and equivalents thereof, but the present disclosure is not limited thereto.

The vertical insulation part 112 is disposed vertically between the metal bodies 111 and serves to electrically insulate a first metal body 111a and a second metal body 111b and to join the first metal body 111a and the second metal body 111b. Accordingly, the first metal body 111a and the second metal body 111b are electrically insulated from each other by the vertical insulation part 112, whereby different voltages may be applied to the first metal body 111a and the second metal body 111b.

The vertical insulation part 112 may be formed in a shape extending in an X-axis direction at the center of the metal bodies 111, or in a shape extending in the X-axis direction at a position deviated from the center of the metal bodies 111.

A cavity 113 may be formed in one side of the metal bodies 111. The cavity 113 may be formed in a tapered shape in which the width thereof gradually decreases downward. The sub-substrate 120 may be provided in the cavity 113. In detail, the cavity 113 may be formed to have a larger size than the sub-substrate 120, and the sub-substrate 120 may be provided at one side in the cavity 113.

A metal layer 116 is formed on the metal bodies 111. The metal layer 116 is for providing electrical connection between the metal bodies 111 and the sub-substrate 120, and may be formed on each of the plurality of metal bodies 111. Herein, the metal layer 116 may be located on an upper surface of each of the metal bodies 111 at a position that does not overlap with the vertical insulation part 112.

The respective metal layers 116 may be formed in a size conforming to that of the sub-substrate 120, and may be formed larger in the size than the sub-substrate 120. Each of the metal layers 116 may have a protrusion 116a which defines a portion of the metal layer 116 at one side thereof. When the sub-substrate 120 is provided in the cavity 113, the respective protrusions 116a of the metal layer 116 may be exposed outside the sub-substrate 120. The metal layers 116 may include two mounting regions. In detail, the metal layers 116 may be divided into a first mounting region in which the sub-substrate 120 is mounted, and a second mounting region which defines the protrusions 116a. Herein, each of the first and second mounting regions may be provided as two regions, with the vertical insulation part 112 interposed therebetween.

The metal layers 116 may be formed to have a quadrangular planar surface, with an area larger than that of the sub-substrate 120, such that edge portions of the metal layers 116 may be externally exposed even after the sub-substrate 120 is mounted. Furthermore, each of the metal layers 116 may be formed in a shape in which one side thereof protrudes. The protrusions 116a of the metal layers 116 may be exposed outside the sub-substrate 120. The shape of the planar surface of the metal layers 116 is not limited thereto, and may be formed in a shape having an arc.

The metal layers 116 may be made of a gold, silver, or copper material having high electrical conductivity, but the material of the metal layers 116 is not limited thereto. Furthermore, the metal layers 116 may be formed by plating, sputtering, or the like.

The sub-substrate 120 is provided in the cavity 113 of the main substrate 110. The sub-substrate 120 is electrically connected to the metal bodies 111 and includes an insulating body 121, a via hole 122 vertically passing through the insulating body 121, and a metal pad 123 connected to the optical element 130.

The insulating body 121 is preferably made of a silicon material, and may be formed to have a predetermined height, with a plurality of via holes 122 formed therein.

The insulating body 121 may be provided on the metal layers 116, and may be joined thereto by bonding with an adhesive means. However, the joining method of the insulating body 121 and the metal layers 116 is not limited thereto.

The insulating body 121 may include the plurality of via holes 122 formed therein. The via holes 122 may be filled with a metal material. For example, the via holes 122 may be filled with gold, silver, copper, or tungsten as a conductive material.

The via holes 122 may be provided in the same number as the metal bodies 111, and the respective via holes 122 may be electrically connected to the different metal bodies 111. Due to the fact that the via holes 122 and the metal bodies 111 are connected to each other, respectively, all the insulating bodies 121 may be electrically connected to the plurality of metal bodies 111.

The via holes 122 are formed in a shape passing through the insulating body 121. Herein, upper sides of the via holes 122 are connected to the metal pad 123, while lower sides of the via holes 122 are electrically connected to the metal layers 116. Accordingly, the voltage of the metal layers 116 may be transferred to the metal pad 123 through the via holes 122. The configuration of the via holes 122 is to join the sub-substrate 120 to the main substrate 110 in a flip chip form. The sub-substrate 120 may be electrically connected to the main substrate 110 through wire bonding without separate provision of the via holes 122. However, a connection configuration through wire bonding requires a separate bonding region that is formed in the metal layers 116 or the metal bodies 111, and thus there is a limitation in increasing the size of the sub-substrate 120.

A plurality of metal pads 123 are provided on the insulating body 121. The metal pads 123 may be configured such that a portion thereof is connected to the via holes 122, and accordingly, the metal pads 123 may be electrically connected to the metal bodies 111. The plurality of metal pads 123 may be provided on the insulating body 121 to be parallel to the vertical insulation part 112, and may be connected to the optical element 130. For example, four metal pads 123 may be arranged in a spaced-apart relationship at a predetermined interval, and two peripheral metal pads 123 may be connected to the via holes 122, respectively. Accordingly, currents flowing through the via holes 122 may be applied to the metal pads 123, respectively.

The two peripheral metal pads 123 are respectively connected to the via holes 122 connected to the different metal bodies 111. The voltage transferred to the two peripheral metal pads 123 may be transferred to central metal pads 123 through the optical element 130.

The optical element 130 is mounted on the sub-substrate 120. The optical element 130 is an element that emits light in response to application of the voltage of the main substrate 110 through the sub-substrate 120, and may be a light-emitting diode (LED). The optical element 130 may be provided as an ultraviolet light-emitting diode (UV LED) for emitting ultraviolet (UV) light.

The optical element 130 may include two electrodes 131, and may be mounted in a flip chip form on the sub-substrate 120. Herein, the electrodes 131 may be electrically connected to the different metal pads 123, respectively.

As illustrated in FIGS. 2 and 3, when the four metal pads 123 are provided on the sub-substrate 120, nine optical elements 130 may be provided in a spaced-apart relationship at a predetermined interval. A plurality of optical elements 130 may be connected to each of the metal pads 123. For example, three optical elements 130 may be connected to one metal pad 123.

Six optical elements 130 may be connected to each of the metal pads 123. For example, three optical elements 130 may be connected to each of the peripheral metal pads 123, and six optical elements 130 may be connected to each of the central metal pads 123. Herein, the electrodes 131 of each of the optical elements 130 may be connected to adjacent metal pads 123, respectively, and thus each of the optical elements 130 may be simultaneously connected to two metal pads 123. The metal pads 123 and the optical elements 130 may be connected in series, parallel, or series-parallel.

The optical elements 130 are mounted in the cavity 113 having a tapered shape in which the width thereof gradually decreases downward. Herein, due to the tapered shape of the cavity 113 of which the width gradually decreases downward, the cavity 113 may include an inclined surface (not illustrated). The inclined surface may function to reflect the UV light irradiated from the optical elements 130.

The light transmission member 140 is provided above the optical elements 130. The light transmission member 140 may be provided in a structure covering the cavity 113, and may be made of a transparent material such that UV light irradiated from the optical elements 130 may be transmitted therethrough. For example, the light transmission member 140 may be made of a quartz material, but the material of the light transmission member 140 is not limited thereto.

Meanwhile, a Zener diode 150 is mounted on the metal layers 116. The Zener diode 150 is a diode for protecting the optical elements 130 from reverse voltage and static electricity, and may be configured such that a first side and a second side thereof are connected to the respective metal layers 116.

In detail, the Zener diode 150 may be provided in any one of the metal bodies 111, and may be electrically connected to a remaining one of the metal bodies 111 through a wire. In other words, the Zener diode 150 may be connected to the metal layers 116 in a vertical shape as illustrated in FIG. 2. Herein, the Zener diode 150 may be mounted on the protrusions 116a of the metal layers 116. The Zener diode 150 may be provided at a position that does not overlap with the sub-substrate 120.

The UV light source 100 as described above may be supported by the support 200.

The support 200 may be formed in a cylindrical shape, and may include a side support 210 supporting the UV light source 100 at an upper edge and a side surface of the UV light source 100 and a lower support 220 supporting the UV light source 100 under the UV light source 100.

The side support 210 may be formed in a cylindrical shape, and may be provided with a seating hole 211 and a plurality of through flow paths 212.

A stepped portion 250 having a predetermined depth may be formed on each of upper and lower surfaces of the side support 210. By forming the respective stepped portions 250 on the upper and lower surfaces of the side support 210, the length of the through flow paths 212 which will be described later may be shortened. By shortening the length of the through flow paths 212, it is possible to more efficiently form the flow of the fluid flowing from the lower flow path 400 to the upper flow path 300.

In the stepped portions 250, the seating hole 211 may be formed through the stepped portions 250, and the plurality of through flow paths 212 may be formed along a circumferential direction of the seating hole 211.

The seating hole 211 may be formed while passing through the side support 210 in a Z-axis direction at the center of the side support 210.

The seating hole 211 may be formed in a shape in which the inner diameter thereof is stepped and gradually decreases from bottom to top. In other words, the width of the inner diameter of the bottom of the seating hole 211 may be larger than that of the top of the seating hole 211.

The UV light source 100 and the lower support 220 may be sequentially seated in the seating hole 211. A first protrusion 230 may be provided above the seating hole 211.

The first protrusion 230 is formed to protrude from the side support 210 toward the inside of the seating hole 211, and serves to prevent the UV light source 100 from being separated upwardly from the support 200.

In detail, the fluid flowing in the UV sterilizer 1 flows in the direction from bottom to top. In this case, the UV light source 100 seated on the support 200 receives a force that pushes the UV light source 100 from bottom to top by the flow direction of the fluid. When the UV light source 100 receives the force by the flow of the fluid, there is a possibility that the UV light source 100 may be separated upwardly from the support 200. Therefore, the first protrusion 230 protruding toward the inside of the seating hole 211 supports an upper surface of the UV light source 100, thereby preventing the UV light source 100 from being separated upwardly from the support 200.

The plurality of through flow paths 212 may be configured so that each of the through flow paths 212 is formed along a peripheral circumference of the seating hole 211. The through flow paths 212 may be formed while passing through the side support 210 in the Z-axis direction. The through flow paths 212 may be formed in arc shapes spaced apart from each other.

The through flow paths 212 serve to allow communication between the lower flow path 400 and the upper flow path 300. In other words, the fluid flows from the lower flow path 400 to the upper flow path 300 through the through flow paths 212.

When the UV light source 100 is seated in the seating hole 211, the lower support 220 may be coupled to the side support 210.

The lower support 220 serves to support the UV light source 100 at a position under the UV light source 100.

A second protrusion 240 may be formed at a lower portion of the lower support 220 to protrude outwardly of the lower support 220. The second protrusion 240 may be coupled to an end of the seating hole 211.

The lower support 220 serves to prevent the fluid from flowing into the UV light source 100 and at the same time serves as a buffer so that a load of the fluid applied to a lower surface of the lower support 220 is not transmitted to the UV light source 100.

In detail, as illustrated in FIG. 1, the UV light source 100 and the lower support 220 are sequentially seated in the seating hole 211. In this case, the lower support 220 is seated under the UV light source 100, while the second protrusion 240 formed at the lower portion of the lower support 220 comes into contact with the end of the seating hole 211. Since the second protrusion 240 is formed to protrude outwardly from a side surface of the lower support 220, when the lower support 220 is seated in the seating hole 211, the interface between the seating hole 211 and the lower support 220 has a stepped shape. Therefore, even if the fluid flows into the gap between the seating hole 211 and the lower support 220, the fluid cannot reach the UV light source 100 due to the stepped interface.

In addition, even if a load of the fluid is applied to the lower surface of the lower support 220 by the flow of the fluid, it is possible to prevent the load of the fluid from being transmitted to the UV light source 100 by supporting the load on a contact surface between the end of the seating hole 211 and the second protrusion 240.

The side support 210 may have a connection hole 213 formed from an outer surface of the side support 210 to the seating hole 211 in an Y-axis direction.

The connection hole 213 connects the outside with the seating hole 211 in which the UV light source 100 is installed so as to discharge heat generated from the UV light source 100 to the outside, thereby preventing the UV light source 100 from thermally expanding and preventing moisture from being generated therein. In addition, the connection hole 213 may be used to provide a wire connected to the UV light source 100.

An O-ring 50 may be provided where two or more members come into contact with each other. In other words, the O-ring may be provided between the upper body 30 and the light source module 10, between the lower body 40 and the light source module 10, between the UV light source 100 and the side support 210, and between the lower support 220 and the side support 210.

Each of the upper body 30, the lower body 40, and the light source module 10 may have a fastening portion (not illustrated) so that the upper body 30, the lower body 40, and the light source module 10 may be detachable from each other. The fastening portion is a portion that may be fastened by various methods, such as bolting, screwing, and the like. For example, each of the upper body 30 and the lower body 40 may have a screw thread formed on an inner surface thereof and the light source module 10 may have a screw thread on an outer surface thereof, so that the upper body 30, the lower body 40 and the light source module 10 may be screwed to each other by rotation.

The O-ring 50 is provided between the members, thereby preventing the fluid from flowing into the gap between the members.

Hereinbelow, a fluid flow of the UV sterilizer 1 according to the first embodiment of the present disclosure will be described with reference to FIG. 1.

As illustrated in FIG. 1, the fluid flows from the lower body 40 to the upper body 30 through the light source module 10. The fluid flows from the first lower flow path 410 to the second lower flow path 420, and the fluid flowing in the second lower flow path 420 flows along a lower surface of the light source module 10 and then flows into the through flow paths 212.

The fluid flowing in the through flow paths 212 flows into the second upper flow path 320, and then is guided to an upper portion of the UV light source 100 by the inner shape of the second upper flow path 320. The fluid guided to the upper portion of the UV light source 100 is sterilized by UV light emitted from the UV light source 100.

The sterilized fluid flows into the first upper flow path 310 and flows out into a flow path in communication with the first upper flow path 310.

In the UV sterilizer 1 according to the embodiment of the present disclosure, by providing the light source module 10 having the UV light source 100 in a flow path along which the fluid flows, it is possible to sterilize the flowing fluid by irradiation by UV light.

In addition, by allowing the fluid to flow while surrounding the UV light source 100, it is possible to expect an effect of naturally removing the heat generated from the UV light source 100.

UV Sterilizer 1′ According to a Second Embodiment of the Present Disclosure

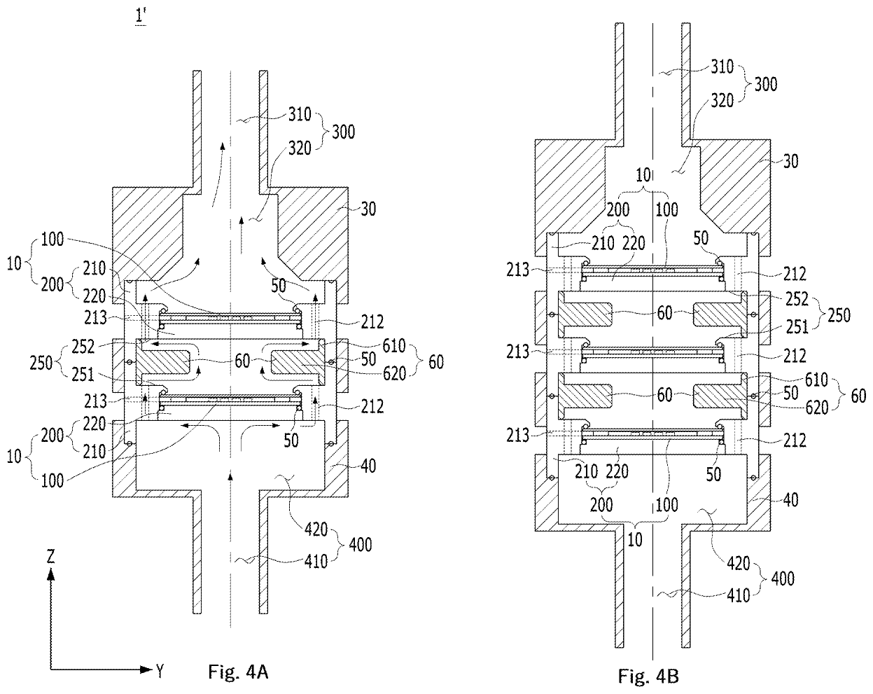

Hereinbelow, a UV sterilizer 1′ according to a second embodiment of the present disclosure will be described with reference to FIGS. 4A and 4B.

Compared to the UV sterilizer 1 according to the first embodiment of the present disclosure, the UV sterilizer 1′ according to the second embodiment of the present disclosure further includes a fluid guide 60, and a plurality of light source modules 10 is provided. Therefore, other configurations of the UV sterilizer 1′ according to the second embodiment of the present disclosure can refer to the description of the UV sterilizer 1 according to the first embodiment of the present disclosure, and redundant descriptions are omitted.

As illustrated in FIGS. 4A and 4B, the UV sterilizer 1′ may include the plurality of light source modules 10.

Two or more light source modules 10 may be provided between an upper body 30 and a lower body 40. In this case, the fluid guide 60 may be provided between the plurality of light source modules 10.

For example, as illustrated in FIGS. 4A and 4B, two or three light source modules 10 may be provided between the upper body 30 and the lower body 40.

When the two light source modules 10 are provided, the light source modules 10 may be stacked and combined in an upper-and-lower direction. In this case, the fluid guide 60 may be provided between the two light source modules 10.

The fluid guide 60 may be composed of a side wall 610 and an inner flange 620.

The side wall 610 may be configured so that upper surface and the lower surfaces thereof are in contact with bottom surfaces of stepped portions of the light source modules 10, respectively. In other words, the upper surface of the side wall 610 may be in contact with a bottom surface of a lower stepped portion 252 of a light source module 10 provided at an upper position (hereinafter referred to as an upper light source module 10), and the lower surface of the side wall 610 may be in contact with a bottom surface of an upper stepped portion 251 of a light source module 10 provided at a lower position (hereinafter referred to as a lower source module 10). Since the upper and lower surfaces of the side wall 610 may be in contact with the bottom surfaces of the stepped portions of the respective light source modules 10, the side wall 610 may be supported and provided between the plurality of light source modules 10.

The inner flange 620 may be formed to extend inwardly from the side wall 610 and to be open. In detail, the inner flange 620 may be formed to extend from the side wall 610 inwardly of the side wall 610. In this case, the inner flange 620 may have a central opening to allow communication between the lower light source module 10 and the upper light source module 10. In other words, a fluid flowing in the lower light source module 10 may flow into the upper light source module 10 through the opening of the inner flange 620.

In the UV sterilizer 1′ according to the second embodiment of the present disclosure, by providing the plurality of light source modules 10, with the fluid guide 60 provided between the plurality of light source modules 10, it is possible to expect a high sterilization effect.

In detail, the fluid flowing in the lower body 40 may flow into the lower light source module 10, and flow into the space between the upper light source module 10 and the lower light source module 10 through flow paths 212. In this case, the flow of the fluid may be guided to the opening of the inner flange 620 by the inner flange 620 of the fluid guide 60. In other words, the fluid flowing in through the lower light source module 10 is guided to an upper portion of the UV light source 100 of the lower light source module 10. Therefore, the fluid may be primarily sterilized by UV light emitted from the lower light source module 10.

The primarily sterilized fluid may flow to a lower portion of the upper light source module 10 through the opening of the fluid guide 60. In this case, since the fluid guide 60 may have the central opening, the fluid may flow into a central portion of a lower surface of the upper light source module 10. The fluid flowing to the lower portion of the upper light source module 10 may flow into through flow paths 212 formed outside the upper light source module 10. The fluid may then flow to an upper portion of the upper light source module 10 through the upper light source module 10, and be secondarily sterilized by UV light emitted from the upper light source module 10.

As described above, in the UV sterilizer 1′ according to the second embodiment of the present disclosure, by providing the plurality of light source modules 10 in a flow path, it is possible to sterilize the fluid flowing along the flow path a plurality of times through the plurality of light source modules 10.

In addition, by installing the fluid guide 60 between the plurality of light source modules 10, it is possible to guide the flow of the fluid is guided toward the light source modules 10, thereby increasing a sterilization effect by UV light and a cooling effect of the UV light sources 100.

UV Sterilizer 1″ According to a Third Embodiment of the Present Disclosure

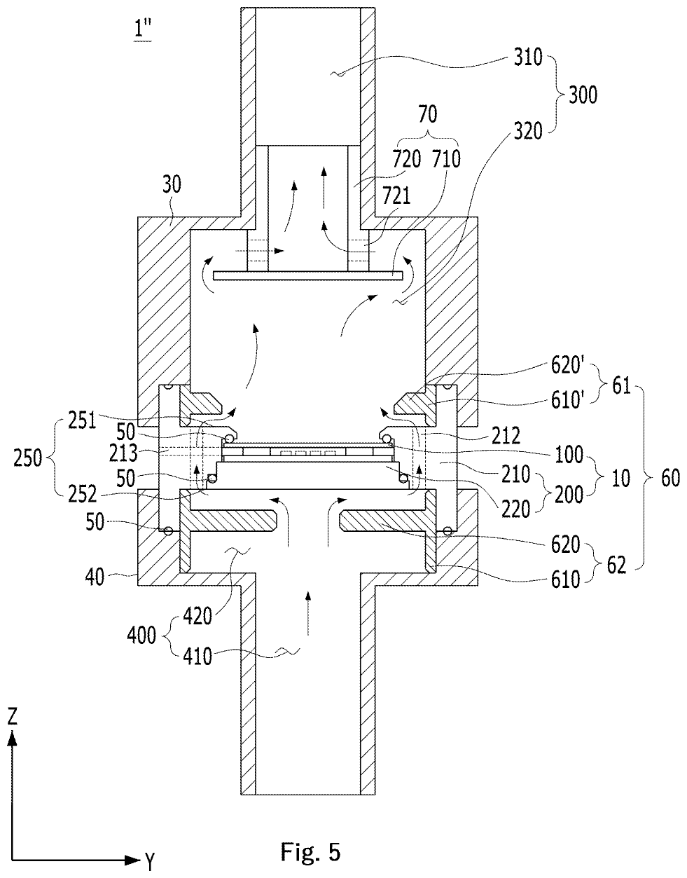

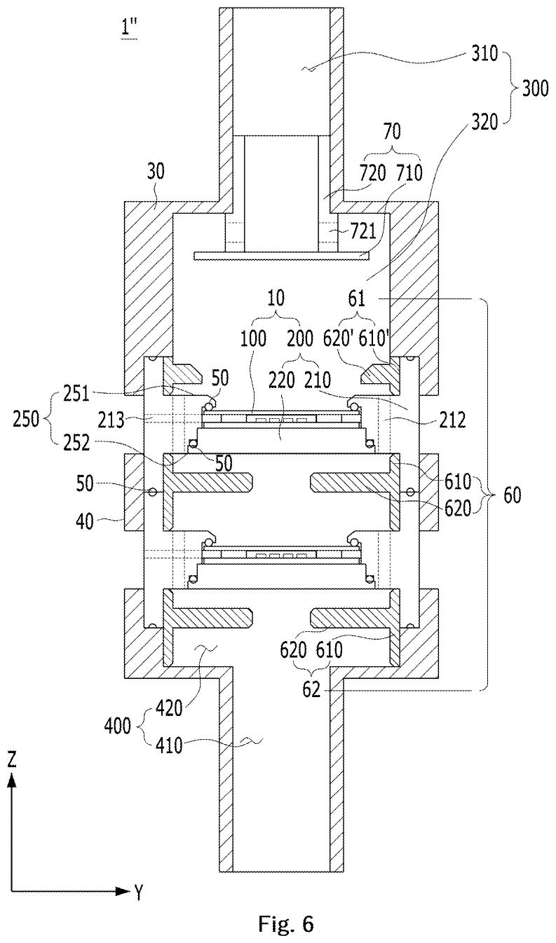

Hereinbelow, a UV sterilizer 1″ according to a third embodiment of the present disclosure will be described with reference to FIGS. 5 and 6.

Compared to the UV sterilizer 1′ according to the second embodiment of the present disclosure, the UV sterilizer 1″ according to the third embodiment of the present disclosure further includes a flow resistor 70 provided in a light source module 10.

Therefore, other configurations of the UV sterilizer 1″ according to the third embodiment of the present disclosure can refer to the description of the UV sterilizer 1′ according to the second embodiment of the present disclosure, and redundant descriptions are omitted.

As illustrated in FIG. 5, the UV sterilizer 1″ according to the third embodiment of the present disclosure may include a fluid guide 60 on at least one of upper and lower portions of the light source module 10, and the flow resistor 70 may be provided in an upper flow path 300.

The fluid guide 60 may be composed of a first fluid guide 61 and a second fluid guide 62.

The first fluid guide 61 may be provided on top of a plurality of light source modules 10. The first fluid guide 61 may have a larger opening than that of the second fluid guide 62. In other words, the area of an opening of an inner flange 620′ of the first fluid guide 61 may be larger than that of an opening of an inner flange 620 of the second fluid guide 62.

The second fluid guide 62 may be provided on bottom of the plurality of light source modules 10 and may be provided between the plurality of light source modules 10. The second fluid guide 62 may have a smaller opening than that of the first fluid guide 61. The second fluid guide 62 serves to guide a fluid flowing from a position below each of the light source modules 10 to a central portion of a lower surface of the light source module 10.

The flow resistor 70 may include a base plate 710 and an extension flow path 720.

The base plate 710 may be formed in a polygonal plate shape such as a square or a circle. The base plate 710 may be made of a material having high reflectivity such as aluminum or an aluminum alloy, but may also be made of a resin-based material. However, when the base plate 710 is made of a material having low reflectivity such as a resin-based material, a lower surface of the base plate 710 may be coated with a material such as aluminum or an aluminum alloy.

An inner surface of a second upper flow path 320 where the base plate 710 is provided may be coated with a material such as aluminum or an aluminum alloy to increase reflectivity.

The base plate 710 may be provided above the light source modules 10, and the lower surface of the base plate 710 and upper surfaces of the light source modules 10 may face each other. The base plate 710 may serve to reflect UV light emitted from the light source modules 10.

The base plate 710 may be provided with the extension flow path 720 extending upwardly from the base plate 710 and having a plurality of holes formed in a side surface thereof.

The extension flow path 720 may have a first end connected to the base plate 710 and a second end open. The second end of the extension flow path 720 may be inserted and/or fastened to a first upper flow path 310 to be fixed. The extension flow path 720 may be fastened to the first upper flow path 310 by various methods, such as bolt fastening, screw fastening, and the like, to be fixed to the first upper flow path 310.

The plurality of holes 721 may be formed in the side surface of the extension flow path 720. The plurality of holes 721 may be formed between the base plate 710 and the first upper flow path 310, may allow communication between the second upper flow path 320 and the extension flow path 720.

In the UV sterilizer 1″ according to the third embodiment of the present disclosure, by installing the fluid guide 60 and the flow resistor 70 to guide the fluid to an upper portion of the light source module 10, it is possible to increase sterilization efficiency by UV light.

In detail, the fluid may flow into a lower body 40 and flow to a lower portion of the light source module 10. In this case, the fluid flowing to the lower portion of the light source module 10 may flow to the central portion of the lower surface of the light source module 10 through the opening of the second fluid guide 62. The fluid may flow to the upper portion of the light source module 10 through flow paths 212 provided outside the light source module 10 along the lower surface of the light source module 10.

The fluid flowing to the upper portion of the light source module 10 may flow along the second upper flow path 320. In this case, the fluid flowing along the second upper flow path 320 is guided to opposite sides of the flow resistor 70 by the base plate of the flow resistor 70. In other words, the fluid may stay between the flow resistor 70 and the upper portion of the light source module 10 by the flow resistor 70 so as to be sterilized by UV light emitted from the light source module 10.

In addition, since the flow resistor 70 may be made of a material that reflects UV light, UV light emitted from the light source module 10 may be reflected on the base plate 710 of the flow resistor 70, thereby irradiating UV light to a wider area to sterilize the fluid.

In addition, since the fluid guide 60 may be provided at a lower portion of the light source module 10, the fluid may be guided to the central portion of the lower surface of the light source module 10 so that the fluid may flow from the central portion of the lower surface of the light source module 10 to peripheral portions thereof, thereby making it possible to effectively cool a UV light source 100.

Meanwhile, as illustrated in FIG. 6, in the UV sterilizer 1″ according to the third embodiment of the present disclosure, the plurality of light source modules 10 may be stacked. In this case, the first fluid guide 61 may be provided on top of the light source modules 10, and a plurality of second fluid guides 62 may be provided on bottom of the light source modules and between the plurality of light source modules 10, respectively. As such, by providing the plurality of light source modules 10 between an upper body 30 and the lower body 40, it is possible to increase a sterilizing effect of the fluid.

As described above, the present disclosure has been described with reference to the exemplary embodiments. However, those skilled in the art will appreciate that various modifications, additions, and substitutions are possible, without departing from the scope and spirit of the present disclosure as disclosed in the accompanying claims.

What is claimed is:

1. A UV sterilizer, comprising:
an upper body having an upper flow path;
a lower body having a lower flow path; and
a light source module provided between the upper body and the lower body,
wherein the light source module comprises:
a UV light source; and
a support supporting the UV light source, and having a through flow path allowing communication between the upper flow path and the lower flow path.

2. The UV sterilizer of claim 1, further comprising:
a fluid guide provided on at least one of upper and lower portions of the light source module,
wherein the fluid guide comprises:
a side wall; and
an inner flange with an opening extending inwardly from the sidewall.

3. The UV sterilizer of claim 1, further comprising:
a flow resistor provided in the upper flow path,
wherein the flow resistor comprises:
a base plate; and
an extension flow path extending upwardly from the base plate, and having a plurality of holes formed in a side surface thereof.

4. The UV sterilizer of claim 1, wherein an upper fastening portion fastened to an upper portion of the light source module is provided at a lower portion of the upper body, and
a lower fastening portion fastened to a lower portion of the light source module is provided at an upper portion of the lower body.

5. The UV sterilizer of claim 1, wherein the through flow path comprises a plurality of through flow paths, and the through flow paths are formed in arc shapes spaced apart from each other.

6. The UV sterilizer of claim 1, wherein the light source module comprises a plurality light source modules, and
a fluid guide is provided between the light source modules.
